(19) United States
(12) Patent Application Publication
(10) Pub. No.: US 2018/0051273 A1
(43) Pub. Date: Feb. 22, 2018

(54) SYSTEM AND METHOD FOR DNA EXTRACTION

(71) Applicants: Molly M. Cadle-Davidson, Geneva, NY (US); Dannielle Loranger, Cortland, NY (US)

(72) Inventors: Molly M. Cadle-Davidson, Geneva, NY (US); Dannielle Loranger, Cortland, NY (US)

(73) Assignee: Syracuse University, Syracuse, NY (US)

(21) Appl. No.: 15/679,369

(22) Filed: Aug. 17, 2017

Related U.S. Application Data
(60) Provisional application No. 62/376,200, filed on Aug. 17, 2016.

(51) Int. Cl. C12N 15/10; B01L 3/00

(52) U.S. Cl. CPC C12N 15/1003; C12N 15/101; B01L 3/502; B01L 2400/0403; B01L 2200/0631; B01L 2300/0832; B01L 2300/087; B01L 2200/141

(57) ABSTRACT

A portable device for extracting DNA from a sample comprising a tissue with a plurality of cells. The device includes an opening configured to receive the sample, a first region in fluid communication with the opening and comprising a plurality of tissue processing elements; a second region having a first dehydrated buffer configured to change a first characteristic of the sample and cause lysis of at least some of the plurality of cells in the sample; a third region having a second dehydrated buffer configured to prevent at least some of a plurality of proteins in the sample from progressing from said third region to the fourth region; a fourth region having a third dehydrated buffer configured to purify the DNA from said sample; and an exit designed to allow for the purified DNA to exit the device.

### CROSS-REFERENCE TO RELATED APPLICATION

This application claims priority to U.S. Provisional Patent Application Ser. No. 62/376,200, filed on Aug. 17, 2016, and entitled “System and Method for DNA Extraction,” the entire disclosure of which is incorporated herein by reference.

### FIELD OF THE INVENTION

The present invention relates to systems and methods for processing a cell sample and extracting DNA for analysis and, more specifically, to a four-stage DNA extraction device.

### BACKGROUND

DNA manipulation is a ubiquitous component of molecular research, medical diagnosis, and a myriad of other applications. Although there is highly sophisticated and efficient equipment for the analysis of DNA, that DNA must first be extracted from biological cells, which can be a limiting factor in DNA analysis. Accordingly, there is high demand for quick and affordable extraction of DNA from a sample. The extraction must obtain sufficient DNA from the sample, and the extract should be of sufficient quality to allow for downstream analysis and manipulation via such sensitive processes such as polymerase chain reaction, among others. Further, the extraction should be designed to be performed rapidly while minimizing the potential for operator error.

Normally, DNA is a highly stable, bio-informative compound that requires specialized equipment and a lab environment to extract in a pure form. Although crude extracts can be made in the field, this process can take considerable time and the resultant product may be unsuitable for downstream fingerprinting or other processes due to remaining cell contaminants. The ability of such contaminants to render DNA useless for forensic application is extreme in plant samples. Although commercial products of extraction of DNA are available, they often cannot be used in the field or are very cumbersome and expensive.

Accordingly, there is a continued need for DNA extraction systems and methods that are both rapid and cost-effective while obtaining sufficient quantities of high quality DNA.

### SUMMARY OF THE INVENTION

The disclosure is directed to devices and methods for extracting DNA. Specifically, a portable device and method for extracting DNA from a sample comprising a tissue with a plurality of cells. According to embodiments described or otherwise envisioned herein, the device includes an opening configured to receive a sample, and then a series of four different regions to process the tissue, lyse the cells, degrade or bind the proteins, and purify DNA, in a sequential order as the sample or its various components are pushed by an external force from one region to the next.

According to an aspect is a portable device for extracting DNA from a sample, the sample being or having tissue with a plurality of cells. The device includes: (i) an opening configured to receive the sample; (ii) a first region in fluid communication with the opening and having a plurality of tissue processing elements; (iii) a second region in fluid communication with the first region, the second region having a first dehydrated buffer configured to change a first characteristic of the sample and cause lysis of at least some of the plurality of cells in the sample; (iv) a third region in fluid communication with the second region, the third region having a second dehydrated buffer configured to prevent at least some of a plurality of proteins in the sample from progressing from the third region to the fourth region; (v) a fourth region in fluid communication with the third region, the fourth region having a third dehydrated buffer configured to purify the DNA from the sample; and (vi) an exit in fluid communication with the fourth region and designed to allow for the purified DNA to exit the device; (vii) wherein application of an external force upon the portable device creates a pressure that pushes at least the DNA in the sample from the opening to the exit.

According to an embodiment, the portable device is a syringe-like device.

According to an embodiment, the plurality of tissue processing elements are shredding elements.

According to an embodiment, the first region further comprises a tissue shredding buffer.

According to an embodiment, the second dehydrated buffer is a protein denaturing buffer.

According to an embodiment, the third region comprises a protein-binding matrix.

According to an embodiment, the fourth region comprises a carbohydrate-binding element.

According to an embodiment, the device also includes a DNA collection module in fluid communication with the exit.

According to an aspect is a method for extracting DNA from a sample comprising a tissue with a plurality of cells includes the steps of: (a) providing a device including: (i) an opening configured to receive the sample; (ii) a first region in fluid communication with the opening and having a plurality of tissue processing elements; (iii) a second region in fluid communication with the first region, the second region having a first dehydrated buffer configured to change a first characteristic of the sample and cause lysis of at least some of the plurality of cells in the sample; (iv) a third region in fluid communication with the second region, the third region having a second dehydrated buffer configured to prevent at least some of a plurality of proteins in the sample from progressing from the third region to the fourth region; (v) a fourth region in fluid communication with the third region, the fourth region having a third dehydrated buffer configured to purify the DNA from the sample; and (vi) an exit in fluid communication with the fourth region and designed to allow for the purified DNA to exit the device; (vii) wherein application of an external force upon the portable device creates a pressure that pushes at least the DNA in the sample from the opening to the exit; (b) placing the tissue into the opening; (c) and applying an external force on the tissue, where the portable device is configured to receive the external force and push the tissue through the first, second, third, and fourth regions.

According to an aspect is a device configured to extract DNA from a sample. The device includes: the system comprising: (i) an opening configured to receive the sample; (ii) a first region in fluid communication with the opening and comprising a plurality of tissue processing elements; (iii) a second region in fluid communication with the first region, the second region comprising a first dehydrated buffer, the first dehydrated buffer configured to change a first characteristic of the sample and cause lysis of at least some of the plurality of cells in said sample; (iv) a third region in fluid communication with the second region, the third region comprising a second dehydrated buffer, the second dehydrated buffer configured to prevent at least some of a plurality of proteins in said sample from progressing from said third region to said fourth region; (v) a fourth region in fluid communication with the third region, the fourth region comprising a third dehydrated buffer, the third dehydrated buffer configured to purify the DNA from said sample; and (vi) an exit in fluid communication with the fourth region and designed to allow for the purified DNA to exit the device.

These and other aspects of the invention will be apparent from reference to the embodiments described hereinafter.

### BRIEF DESCRIPTION OF THE DRAWINGS

The present invention will be more fully understood and appreciated by reading the following Detailed Description in conjunction with the accompanying drawings, in which:

FIG. 1 is a flowchart of a method for extracting DNA from a sample in accordance with an embodiment.

FIG. 2 is a schematic representation of a device for extracting DNA from a sample in accordance with an embodiment.

FIG. 3A is a schematic representation of a device for extracting DNA from a sample, prior to application of an external force, in accordance with an embodiment.

FIG. 3B is a schematic representation of a device for extracting DNA from a sample, after application of an external force, in accordance with an embodiment.

### DETAILED DESCRIPTION

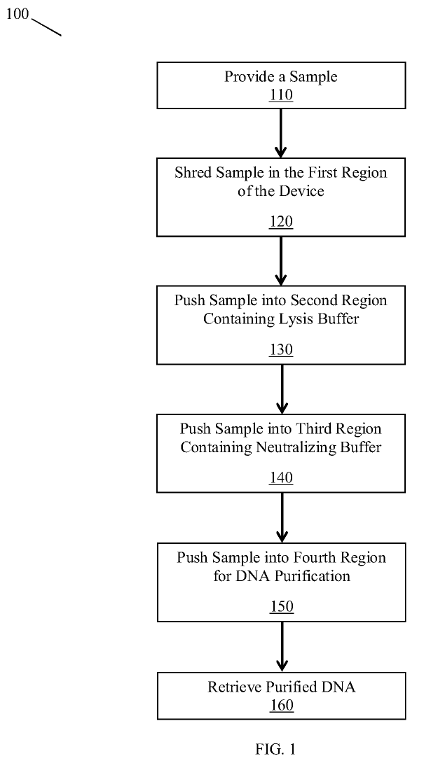

Referring now to the drawings, wherein like reference numerals refer to like parts throughout, there is seen in FIG. 1 a method 100 for extracting DNA from a sample using a four-stage extraction device. According to an embodiment, the extraction device is a syringe used for processing a cell sample and extracting DNA for analysis. The syringe can comprise four stages: (1) a first stage designed or configured for shredding or shearing of an introduced sample; (2) a first buffering stage comprising a prepopulated dehydrated buffer for the sample; (3) a second buffering stage comprising a dehydrated buffer configured to process proteins in the sample; and (4) a third buffering stage comprising a DNA cleanup buffer. According to an embodiment, the size of the device can depend on the size of the sample from which DNA is to be extracted with very small samples could be processed using a device no larger in diameter than a pencil, for example, while larger samples may require larger diameters. Alternatively, the device may be configured to accept and process samples ranging from very small to very large. For samples that exceed the capacity of the device, the sample could undergo pre-processing.

At step 110 of the method, the sample is provided. The sample can be any sample containing DNA, such as a leaf or other plant tissue, a touch sample swab, or other DNA-containing material. According to an embodiment, the sample is obtained directly in the field for which rapid identification or other analysis is required. The sample is preferably placed in the top of the device, such as syringe barrel or similar structure, among other possibilities.

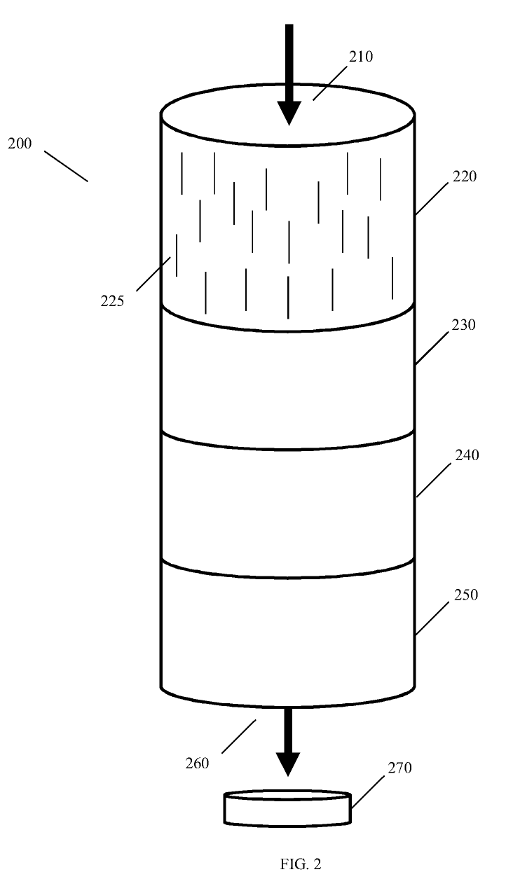

At step 120 of method 100 depicted in FIG. 2, the sample is pushed through and thus processed by a first region of the extraction device. According to an embodiment this region has a matrix of passages with sharp edges such that when material is forced through it will be shredded. The effective pore size of the region is small enough to cause cell breakage. Should additional shredding be required, a mechanical aspect powered by the action of the plunger can be added to this region. Possible implementations of this could be a scissor-like or spinning blade action that will actively slice or chop the sample as it is pushed through the region. According to an embodiment this region can comprise a tissue shredding buffer to facilitate processing and/or facilitate downstream steps.

At step 130 of method 100 depicted in FIG. 2, the sample is pushed into a second region of the device. According to an embodiment this region is a matrix possessing dehydrated buffer in its pores or surface. When the sample passes through this region the buffer is rehydrated thus causing a pH change that denatures the sample. This results in cell lysis and release of cell contents.

At step 140 of method 100 depicted in FIG. 2, the sample is pushed into a third region of the device. According to an embodiment this region is a matrix possessing a dehydrated neutralizing buffer. According to an embodiment this region binds or otherwise neutralizes or processes proteins. For example, the denaturing buffer from the second region can be exchanged and cell proteins can be retained as the sample is pushed through the third region.

At step 150 of method 100 depicted in FIG. 2, the sample is pushed into a fourth region of the device. According to an embodiment this region comprises a matrix with dehydrated buffer that serves as a buffer exchange and purification step. For example, carbohydrates can be retained through binding with fixed carbohydrate-binding modules (e.g., modified glycoside hydrolases). Thus, only purified DNA passes through the fourth region.

At step 160 of method 100 depicted in FIG. 2, the purified DNA is pushed pass the fourth region. According to one embodiment, the purified DNA can be emitted into a collection component attached to the device. According to another embodiment, the purified DNA can be collected in a tube, vial, or other collection device. In addition, there are many other ways to collect the purified DNA.

Accordingly, the four regions function to extract DNA in the following way, pursuant to one embodiment. A sample (leaf or other plant tissue; touch sample swab; other DNA-containing material) is placed in the top of the syringe barrel. The syringe plunger is pushed into the barrel which releases a volume of tissue shredding buffer necessary to move the sample through the first region. This volume is dependent on the diameter of the syringe being used but is no more than the bed volume of any one of the four regions. The sample is shredded by passing over the sharp edges in the first region which may vary in arrangement and/or mechanism to produce the most effectively disrupted sample. The plunger is pushed further which causes the sample to move into the second region of the barrel and large cell/tissue components to be left behind in the first region. Buffer moving into the second region rehydrates the denaturation buffer and causes a pH change and buffer exchange that lyses the sample cells. Continued pressure on the plunger moves the sample into the third region where rehydration again occurs, this time with the purpose of neutralizing the extraction buffer. Additionally, proteins are bound to the matrix by the presence of fixed poly-L-lysine or another similar protein binding substance. As the plunger is pressed further, proteins are retained by the first region and the balance of the sample moves into the fourth region. A purification buffer is rehydrated by the sample moving into this region and any remaining carbohydrates are removed by their interaction with fixed carbohydrate binding modules such as glyocoside hydrolases. As the plunger pushes the sample out of this region, purified DNA is expelled from the syringe into either an attached receptacle or directly into a downstream DNA analysis device. Such devices may be for PCR, HRM, microfluidics applications, or sequencing, to name a few.

According to an embodiment, the time required to push a sample through the DNA extraction syringe will be approximately five minutes or less. Short pauses may be necessary for protein and carbohydrate binding in the third and fourth regions, and these can be clearly marked on the barrel of the syringe, for example. Other mechanisms to properly control the flow rate through the device are also possible.

FIG. 2 is a schematic representation of a four-stage extraction device 200 utilized to extract DNA from a sample, according to an embodiment. Device 200 comprises a sample entrance 210, a first stage 220, a second stage 230, a third stage 240, a fourth stage 250, and a sample exit 260. According to one embodiment, four-stage extraction device 200 is similar in shape and size to a syringe barrel.

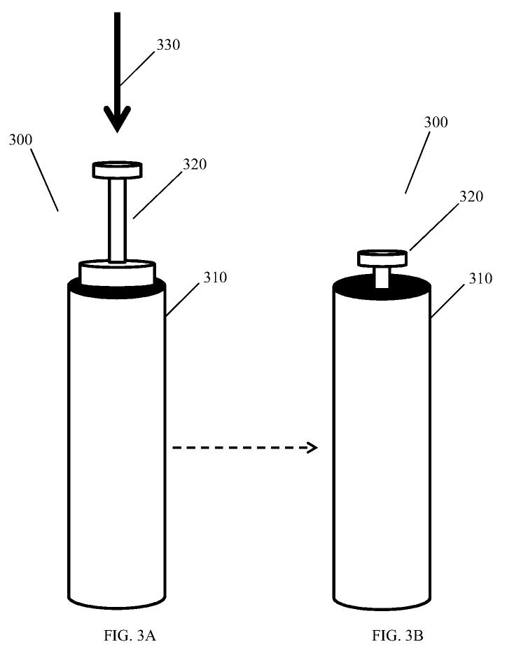

Sample entrance 210 can be as simple as an open top portion of the extraction device, although it is preferably covered when not in use in order to prevent contamination. The entrance can also be an initial stage of processing, such as a grinder, shredder, mixer, or other processing component. The entrance is preferably sized to allow a plunger (such as element 330 in FIG. 3) to enter the device and/or push the sample through the extraction device.

First stage 220 of four-stage extraction device 200 receives the sample and, according to one embodiment, can comprise a matrix of passages with sharp edges such that when material is forced through it will be shredded. The effective pore size of the region is small enough to cause cell breakage. The first stage 220 can also or alternatively include a mechanical aspect for breaking down the sample, such as a blade or series of blades, among other mechanical components. According to an embodiment this region can comprise a tissue shredding buffer to facilitate processing and/or facilitate downstream steps.

Second stage 230 of four-stage extraction device 200 receives the processed sample from the previous stage. According to an embodiment second stage 230 comprises a matrix possessing dehydrated buffer in its pores or surface. When the sample passes through this region the buffer is rehydrated thus causing a pH change that denatures the sample. This results in cell lysis and release of cell contents.

Third stage 240 of four-stage extraction device 200 receives the processed sample from the previous stage. According to an embodiment third stage 240 comprises a matrix possessing a dehydrated neutralizing buffer. According to an embodiment this region binds or otherwise neutralizes or processes proteins. For example, the denaturing buffer from the second region can be exchanged and cell proteins can be retained as the sample is pushed through the third region.

Fourth stage 250 of four-stage extraction device 200 receives the processed sample from the previous stage. According to an embodiment fourth stage 250 comprises a matrix with dehydrated buffer that serves as a buffer exchange and purification step. For example, carbohydrates can be retained through binding with fixed carbohydrate-binding modules (e.g., modified glycoside hydrolases). Thus, only purified DNA passes through the fourth region.

The purified DNA can then leave the extraction device via exit 260. Exit 260 may be as simple as an opening, or can be a collection device or component 270. The collection device or component 270 can be, for example, a removable or operable component of the extraction device, or can be a separate device designed to collect DNA or a sample, such as an Eppendorf tube or similar collection device. The collection device or component 270 can include a storage buffer designed to maximize storage of the DNA or can include an analyte designed for immediate analysis of the purified DNA.

FIG. 3 is a schematic representation of a four-stage extraction device 300 in a first configuration (A) and a second configuration (B). Device 300 comprises a processing region 310, depicted as a barrel in the figure, and an actuator 320 depicted as a plunger in the figure. To process a sample, the sample is placed into or near the entrance to the processing region 310 when the device is in first configuration (A), the actuator 320 is placed in the processing region, and a force is applied in the direction of arrow 330 in FIG. 3. The force pushes the actuator through the processing region, forcing the sample through the processing region, and changing the device to second configuration (B).

This extraction system or method could be used for rapid, in-the-field processing of samples for medical applications, for sample identification purposes, or for pathogen identification (e.g., extracting DNA from a field sample to determine whether anthrax is present), among many other uses.

Although the present invention has been described in connection with a preferred embodiment, it should be understood that modifications, alterations, and additions can be made to the invention without departing from the scope of the invention as defined by the claims.

What is claimed is:

1. A portable device for extracting DNA from a sample comprising a tissue with a plurality of cells, the device comprising:
an opening configured to receive the sample;
a first region in fluid communication with the opening and comprising a plurality of tissue processing elements;
a second region in fluid communication with the first region, the second region comprising a first dehydrated buffer, the first dehydrated buffer configured to change a first characteristic of the sample and cause lysis of at least some of the plurality of cells in said sample;
a third region in fluid communication with the second region, the third region comprising a second dehydrated buffer, the second dehydrated buffer configured to prevent at least some of a plurality of proteins in said sample from progressing from said third region to said fourth region;
a fourth region in fluid communication with the third region, the fourth region comprising a third dehydrated buffer, the third dehydrated buffer configured to purify the DNA from said sample; and
an exit in fluid communication with the fourth region and designed to allow for the purified DNA to exit the device;
wherein application of an external force upon the portable device creates a pressure that pushes at least the DNA in the sample from the opening to the exit.

2. The extraction device of claim 1, wherein the portable device is a syringe-like device.

3. The extraction device of claim 1, wherein the plurality of tissue processing element are shredding elements.

4. The extraction device of claim 1, wherein the first region further comprises a tissue shredding buffer.

5. The extraction device of claim 1, wherein the second dehydrated buffer is a protein denaturing buffer.

6. The extraction device of claim 1, wherein the third region comprises a protein-binding matrix.

7. The extraction device of claim 1, wherein the fourth region comprises a carbohydrate-binding element.

8. The extraction device of claim 1, further comprising a DNA collection module in fluid communication with the exit.

9. A method for extracting DNA from a sample comprising a tissue with a plurality of cells, the method comprising the steps of:
providing a portable device comprising: (i) an opening configured to receive the sample; (ii) a first region in fluid communication with the opening and comprising a plurality of tissue processing elements; (iii) a second region in fluid communication with the first region, the second region comprising a first dehydrated buffer, the first dehydrated buffer configured to change a first characteristic of the sample and cause lysis of at least some of the plurality of cells in said sample; (iv) a third region in fluid communication with the second region, the third region comprising a second dehydrated buffer, the second dehydrated buffer configured to prevent at least some of a plurality of proteins in said sample from progressing from said third region to said fourth region; (v) a fourth region in fluid communication with the third region, the fourth region comprising a third dehydrated buffer, the third dehydrated buffer configured to purify the DNA from said sample; and (vi) an exit in fluid communication with the fourth region and designed to allow for the purified DNA to exit the device;
placing the tissue into the opening;
applying an external force on the tissue, wherein the portable device is configured to receive the external force and push the tissue through the first, second, third, and fourth regions.

10. A device configured to extract DNA from a sample, the device comprising:
an opening configured to receive the sample;
a first region in fluid communication with the opening and comprising a plurality of tissue processing elements;
a second region in fluid communication with the first region, the second region comprising a first dehydrated buffer, the first dehydrated buffer configured to change a first characteristic of the sample and cause lysis of at least some of the plurality of cells in said sample;
a third region in fluid communication with the second region, the third region comprising a second dehydrated buffer, the second dehydrated buffer configured to prevent at least some of a plurality of proteins in said sample from progressing from said third region to said fourth region;
a fourth region in fluid communication with the third region, the fourth region comprising a third dehydrated buffer, the third dehydrated buffer configured to purify the DNA from said sample; and
an exit in fluid communication with the fourth region and designed to allow for the purified DNA to exit the device.

11. The device of claim 10, wherein the portable device is a syringe-like device.

12. The device of claim 10, wherein the plurality of tissue processing element are shredding elements.

13. The device of claim 10, wherein the first region further comprises a tissue shredding buffer.

14. The device of claim 10, wherein the second dehydrated buffer is a protein denaturing buffer.

15. The device of claim 10, wherein the third region comprises a protein-binding matrix.

16. The device of claim 10, wherein the fourth region comprises a carbohydrate-binding element.

17. The device of claim 10, further comprising a DNA collection module in fluid communication with the exit.
